(19) United States
(12) Patent Application Publication
(10) Pub. No.: US 2011/0077549 A1
(43) Pub. Date: Mar. 31, 2011

(54) SUPPORT EQUIPMENT FOR MEASUREMENT OF THE ACTIVITY OF THE OPTIC NERVE

(75) Inventors: Ryuhei Kitai, Fukui (JP); Kotaro Nakano, Fukui (JP); Atsuji Masuda, Fukui (JP); Takaharu Tsutani, Fukui (JP); Masukiyo Tsutani, Fukui (JP)

(73) Assignee: UNIVERSITY OF FUKUI, FUKUI-CITY (JP)

(21) Appl. No.: 12/737,047

(22) PCT Filed: Jun. 11, 2009
(86) PCT No.: PCT/JP2009/002634

§ 371 Date: Dec. 3, 2010

(30) Foreign Application Priority Data
Jun. 13, 2008 (JP) 2008-155732

(51) Int. Cl. A61B 3/00

(52) U.S. Cl. 600/558

(57) ABSTRACT

The invention provides easy handling and low cost-support equipment for measuring optical nerve-activity which is less stressful for subjects or doctors and the like. The support equipment, which provides light stimulation for subject eyes with its main body 10 fixed to the subject-head region and measures the subject optical nerve-activity, comprises a thin walled-main body 10 with a plurality of optical fibers 11 joined in the width direction; light emission regions 13a, 13b at least one of which is positioned in the main body aligned to the subject eye-position(s) and composed of a plurality of light leak parts 11c formed in peripheral surfaces of the optical fibers 11; and a connector 12a for connecting with a light emission device 12 providing light for each of the plurality of optical fibers.

### FIELD OF THE INVENTION

The invention relates to a support equipment for measuring optical nerve-activity used for measuring subject-optical nerve-activity by providing optical stimulation to the subject eyes.

### DESCRIPTION OF THE RELATED ART

As for examples about devices of the kind, devices are known which are used for judging whether diseased sites are to be removed or judging whether apparatuses are adequate for removing diseased sites in cerebral nerve-surgical operations under anesthesia by monitoring brain waves along with light-irradiation to eyes of patients (subjects). (Refer to the patent document 1 or 2 as the examples.) In addition, in devices of this family, support equipments which are described in the patent document 3 or 4 are known as the ones providing light stimulation to human subject-eyes.

Patent document 1: Japanese Unexamined Patent Application Publication No. 2003-135414
Patent document 2: Japanese Unexamined Patent Application Publication No. 2006-280421
Patent document 3: Japanese Unexamined Patent Application Publication No. 2003-199722
Patent document 4: Japanese Unexamined Patent Application Publication No. 2007-185326

A main part of the support equipments described in the above document 3 or 4 is a goggle-shape-body made of silicon rubber or plastic and the like, which makes the main body hard and pressing subject-eyeballs with such a main body for a long time increases burdens on the subject. In addition, the design of the goggle shape-main body in the above document 3 or 4 makes the main body thick and heavy and requires fixing of the main body to the subject-head region with high sealing strength-adhesive tape, which further increases pressure to the subject-eyeballs. Furthermore, the heaviness of the main body or cables makes the main body-position easily shifted during long time-operations. Moreover, the main body-position is easily shifted due to action of the cable-weight to the main body along with changes of the subject body-positions or touches of the cables by doctors or nurses during the operation. Furthermore, the measurement device and the light emission device are set away from the main body which is fixed to the subject-head region and the cables for connecting them has high rigidity, which causes detaching of the main body from the subject-head region during the operation. In the face of the above mentioned problems, the present invention was intended to provide an easy handling and low cost-support equipment for measuring optical nerve-activity which solves all of the above described-problems and makes the burdens on the subjects and doctors extremely small.

### SUMMARY OF THE INVENTION

The present invention was designed in the face of the problems mentioned in the Description of the Related Art. In accordance with the present invention, as described in the claim 1, a support equipment comprises a main body which is thin walled with a plurality of optical fibers joined in the width direction; light emission regions at least one of which is positioned in the main body aligned to the subject eye-position(s) and composed of a plurality of light leak parts formed in peripheral surfaces of the optical fibers; and a connector which is set in at least one ends of the optical fibers for connecting with a light emission device providing light for each of the plurality of optical fibers.

The main body in the above structure is formed to be thin walled-strip-shaped, which enables the main body in the support equipment for measuring optical nerve-activity to be lightweight. In accordance with the present invention, it is preferable to use small diameter-optical fibers. More specifically, as described in the claim 2, it is preferable to use flexible optical fibers whose diameter is 0.5 mm or less. As for the main body including the optical fibers, the thickness may be 1 mm or less and the weight may be 50 g or less/m, or 30 g/m preferably. Various sorts of materials such as resin film or paper (Japanese paper) may be employed as the main body-materials if they are thin walled, lightweight, flexible, and have hard-to-rupture-solidity and they can join the plurality of optical fibers. In accordance with the present invention, as described in the claim 3, it is favorable to use the main body which is made of a cloth whose warp threads in the longitudinal direction are partially or all replaced with the above mentioned-optical fibers. In this case, the cloth may be woven with the weft-threads less densely so as to prevent the optical fibers from warping with the weft threads. Such a weaving manner can increase the light-propagation-efficiency.

The above mentioned-plurality of light leak parts formed in the optical fibers should be the ones which irradiate a portion of light propagated in the optical fibers to the subject-eyes and whose lighting intensity is enough to make the subject brain wave-changes appear in a monitor along with the light stimulation. In accordance with the present invention, as described in the claim 4, the light emission regions which evenly produce the light across the subject eyes with sufficient light intensity were produced by making the light leak parts with acute angle-incisions in the optical fiber-peripheral surfaces at various directions and intervals. Furthermore, In accordance with the present invention, as described in the claim 5, melting and cutting of the optical fibers whose one ends were bundled enabled the light from the light emission-means to propagate efficiently. In this case, surfaces of the melted and cut-one ends of the bundled-optical fibers may be polished.

In accordance with the present invention, as described in the claim 6, when a plurality of the light-emission-regions are made in a single main body, the light leak parts in the plurality of optical fibers positioned in parallel in one of the light emission regions should be formed in every other or every plurality of the optical fibers alternately with those in the other light emission region. In addition, in accordance with the present invention, as described in the claim 7, it is preferable the support equipment includes a plurality of the main bodies. This is because layering of the plurality of main bodies can provide sufficient light intensity when the light intensity provided by the single main body is insufficient. In this case, it is preferable to layer the plurality of main bodies keeping the light propagation directions in the optical fibers same so that one ends of the layered main bodies are bundled only on one side of the subject-head region for avoiding the main bodies from being an obstacle for doctors and others in medical front such as operations and the like.

In the support equipment for measuring optical nerve-activity in accordance of the present invention, the optical fibers are joined in the main body which is thin-walled, lightweight, and flexible. Then, the main body is easily attached to the subject-head region; therefore, attaching of the main body to the subject-head region does not cause a long time-pressure to the subject-eyeballs. In addition, just attaching the main body to the subject-head region can fix its position; accordingly, attaching of the main body to the subject-head region does not cause the pressure increase to the eyeballs. Furthermore, the main body is extremely lightweight; therefore, its position-off cannot occur during the long time-operations or its detachment can hardly occur during the operations. Moreover, employing the optical fiber-woven-cloth makes it possible to mass-produce the support equipment for measuring optical nerve-activity at low cost. Thus, the support equipment for measuring optical nerve-activity in accordance of the present invention can lessen the burdens on either the subjects such as patients and the like or doctors and the like and provide the easy handling-support equipment for measuring optical nerve-activity at low cost. Furthermore, the main body is a single use-type and the feature of the support equipment can provide the support equipment for measuring optical nerve-activity with high level-safety.

### DESCRIPTION OF THE PREFERRED EMBODIMENTS

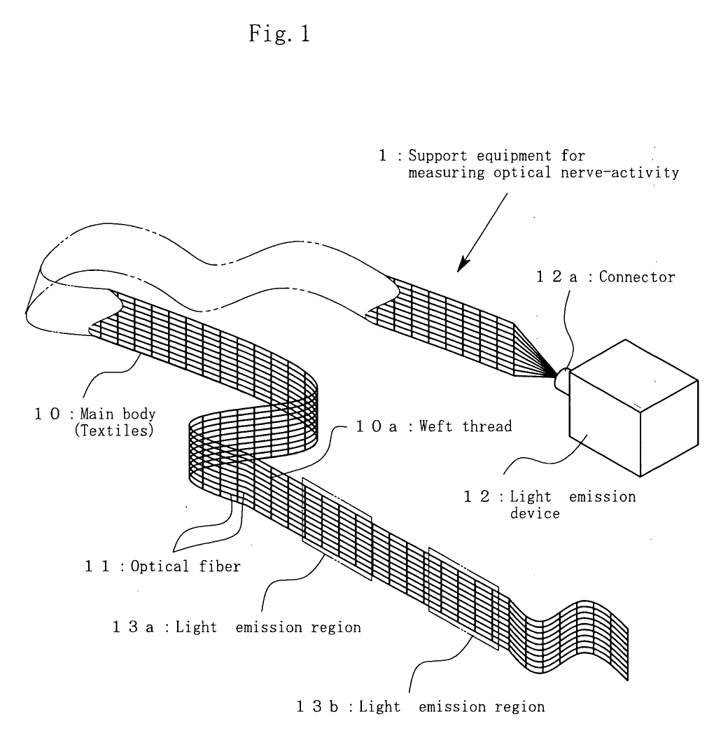

Details on the best mode for carrying out the invention are explained referring to the figures as follows. The FIG. 1 is an oblique perspective-outline drawing for explaining the first embodiment of the support equipment for measuring optical nerve-activity of the present invention. The main body 10, which takes a thin-walled, flexible, and elongated belt-form, is a main formation element of the support equipment for measuring optical nerve-activity. The main body 10 is formed with a plurality of the optical fibers 11 arranged in parallel in the longitudinal direction and joined in the width direction. One ends of the optical fibers 11, which are exposed from the main body 10, are bundled and inserted in a connector 12a and connected or disconnected at will with a light emission device 12 through the connector 12a. Then, the main body 10 used in a surgery and the like can be detached from the light emission device 12 at the connector 12a and disposed at a single use and replaced with the new one in the next surgery.

[Main Body and Optical Fiber] Preferably, the main body 10 should be lightweight, flexible, and have hard-to-rupture-solidity wherein the plurality of optical fibers 11 should be arranged in parallel and joined. The main body in this embodiment is a cloth composed of warp-threads and weft-threads and some or all of the warp-threads woven in the longitudinal direction are replaced with the optical fibers 11. As for the optical fibers 11 as the warp-threads in the main body, there is no particular restrictive condition on the diameter or the number of the used-optical fibers if they do not decrease the main body-flexibility and they provide light intensity required in supporting for measuring optical nerve-activity. It is preferable the material of the optical fibers 11 is plastic which is high flex-resistant and easy to handle. It is also preferable the optical fiber 11-diameter is 0.5 mm or less. For example, it is preferable to use polymethyl acrylate type, polyethyl acrylate type, or polystyrene type-optical fibers whose diameter is approximately 0.25 mm and arrange them in the density of 20 fibers/cm˜40 fibers/cm in the main body. Needless to add, the optical fibers whose diameter is less than 0.25 mm may be used.

The above mentioned-optical fiber-woven cloth is publicly known in such as the Japanese Unexamined Patent Application Publication No. 62-192701, or the Japanese Unexamined Patent Application Publication No. 2007-169807 by Tsutani textile Co., Ltd. who is one of the applicants of the present invention. In order to increase efficiency of the light transmission in the optical fibers 11 as much as possible, it is preferable to use the optical fibers 11 whose flexure due to the weft-threads 10a is as small as possible. As for such optical fiber-woven cloth or the woven method, the Japanese Unexamined Patent Application Publication No. 2008-40046 by the above applicant of the present invention is cited as the example explaining the densely-arranged-optical fibers 11 joined with extra thin warp-threads. In order to set the connector 12a for the optical fibers 11 or to avoid the connector 12a from detaching from the main body 10 easily during the operations, it is preferable the main body 10 is woven with a shuttle or needle-weaving machine and the weaving makes the main body-both selvages robust.

There is no particular restrictive condition on materials, diameter, or woven-density and the like of the weft-threads 10a if they can maintain the main body 10-flexibility and join the optical fibers 11 in the width direction. However, the weft-threads 10a should be preferably non-conductive so as to prevent noise-generation in the measurement device. The weft threads 10a satisfying the above conditions may include; polyethylene terephthalate; PTT(polytrimethylene terephthalate); polyester series fibers such as PBT (polybutylene terephthalate) and the like; nylon (polyamide fibers); aramid (aromatic series polyamide fibers); polyolefin series fibers such as polypropylene or polyethylene and the like; synthetic fibers such as acrylic and the like; chemical fibers such as rayon or acetate and the like; and natural fibers such as cotton, hemp, wool, or silk and the like.

The specific examples of the weft-thread 10a are polyester series fibers as multi-filament whose thickness is approximately 22 dtx˜56 dtx or 2 f˜24 f and whose twisted frequency is 100 t/m or less or single covering textured yarn whose core thread is polyurethane elastic yarn (33 dtx/3 f) and whose kite thread is nylon series draw texturing yarn (Pu33x/Ny33). Preferably, the style of weaving the above mentioned-cloth with warp threads (Optical fibers 11) and the weft-threads 10a should be a plain weave so as to lessen the optical fibers 11-horizontal shift as much as possible. This plain weave joins the optical fibers 11 firmly. It is preferable the weft-threads 10a are woven less densely as approximately 7 threads˜9 threads/cm so as to lessen flexure of the optical fibers 11 with the weft-threads 10a. The specific dimensions of the main body 10 made of the cloth are, for example, the width is approximately 30 mm˜45 mm, the length is approximately 1.5 m˜3 m, and the thickness is 1.0 mm or less. (Preferably, the thickness should be approximately 0.3 mm˜0.8 mm. It is acceptable to make the thickness 0.3 mm or less by using small diameter-optical fibers 11.) The cloth-weight should be 50 g/m or less, preferably 30 g/m or less, and more preferably approximately 7.3 g/m˜20 g/m.

A couple of the light emission regions 13a, 13b are formed in the main body 10 with them aligned with the subject-eye-positions. Positions of the light emission regions. 13a, 13b are beforehand set at the subject-eye-positions. There is no significant individual variation in human eye-positions, although there is slightly between an adult and a child. Then, preparation of the main body 10 for an adult only and for a child only respectively in which the light emission regions 13a, 13b are set at the average positions with some extent-margins.

[Light Emission Region and Light Leak Part] Each of the light emission regions 13a, 13b is composed of a group of the plurality of light leak parts 11c formed in the optical fibers 11. It is preferable the light intensity from across the right and left-light emission regions 13a, 13b should be as even as possible. The FIG. 2 relates to an example of the light leak parts 11c and the FIG. 2(a) is a partially-enlarged cross section diagram of the optical fiber 11 in which the light leak parts 11c are formed. The FIG. 2(b) is a partially-enlarged-plain view of the optical fiber 11 in which the light leak parts 11c are formed. Generally, the optical fiber 11 is composed of a core 11a and cladding 11b. The light leak parts 11c are made by partially making incisions in the cladding 11b developing into the core 11a. This enables some of the light propagated in the optical fibers 11 to divert the transmitted light-directions to subject-eye E. If the above described results are obtained, there is no particular limit on forms of the light leak part 11c. Formation of the light leak parts 11c can be made by publicly known methods such as physical processes with polishing agents such as files, knives, sand-blast, or sand-paper and the like, thermal processes such as heat cut or hot stamp and the like, laser processing with ultraviolet rays and the like or chemical treatment processes and the like.

In this embodiment, a blade such as a cutter and the like is pressed against the optical fiber 11-surface so as the blade-incising direction to meet the optical fiber 11-axis line and the incision with the blade is made in the cladding 11b developing into the core 11a. The cross section-shapes of the light leak parts 11c formed by the above-procedure make acute angles as shown in the FIG. 2(a). It is preferable to be various in intervals, orientations, and depths of the light leak parts 11c as shown in the FIG. 2(a) and (b). Thus, a group of the light leaked from each of the light leak parts 11c in various directions are provided to the subject eyes as evenly-eradiated-light. It is preferable to form the light leak parts with the light being propagated in the optical fibers 11 while checking the intensity of leaked-light from the light leak parts 11c. In addition, it is preferable to form the light leak parts after the optical fibers 11 being woven in the main body 10. In order to make the light leak part 11c-formed positions easy to recognize, it is preferable to use easily-recognizable-weft threads such as dyed threads (dyed-in-threads) or spin dyeing threads and the like.

The plurality of light leak parts 11c are formed in each of the optical fibers in a direction along with the optical fiber 11-axis line. The light leak part-positions are chosen for each of the optical fibers 11 so as the light intensity of the right and left-light emission regions 13a, 13b to be even. The FIG. 3 is a plain view of an example of positioning of the plurality of light leak parts composing the light emission regions 13a, 13b. The light leak part 11c-formed positions are indicated in ovals in the FIG. 3. The plurality of light leak parts 11c which are formed in a direction along with the optical fiber 11-axis line are included in each of the ovals indicated in the FIG. 3. This figure shows the plurality of light leak parts 11c in one of the light emission regions 13a are positioned in every other optical fiber alternately with those in the other light emission region 13b. This positioning of the light leak parts 11c can provide almost even light intensity from the right and left-light emission regions 13a, 13b. For the sake of convenience in the drawing preparation, the light leak parts 11c in one of the light emission regions 13a are positioned in every other optical fiber alternately with those in the other light emission region 13b; however, the light leak parts 11c should be preferably positioned in every plurality of the optical fibers; for example, every 10 optical fibers˜20 optical fibers, in one of the light mission regions 13a alternately with those in the other light emission region 13b.

[Light Emission Device] Publicly known-light emission devices may be used if they can provide irradiation light for the plurality of optical fibers 11 simultaneously. Light entrance surfaces of the optical fiber 11-ends wherein the light from the light emission device 12-light source is provided should be preferably finished as flat as possible for enhancing the light transmission efficiency. In this embodiment, the flatness in the optical fiber 11-ends is obtained by bundling the plurality of optical fibers 11 and melting and cutting them. In this case, the melted and cut-optical fiber 11-end-surface may be polished.

[Fixing of the Main Body 10] After the light emission regions 13a, 13b of the main body 10 are positioned with the subject-eyes E, E, the main body 10 is fixed to subject-head region with adhesive tape for medical use or low-skin irritation-adhesive. When adhesive tape for medical use is employed, the adhesive tape is sealed over the main body 10 across the subject-head region to fix the main body 10 to the subject-head region. When the low-skin irritation-adhesive is used, the adhesive should be preferably colorless and transparent not to obstruct the light irradiation from the light leak parts 11c. As shown in the FIG. 3, fixing parts 14, 14 projecting from the light emission regions 13a, 13b may be prepared and the adhesive may be applied to the fixing parts in advance.

### Second Embodiment

In the second embodiment of the present invention, the FIG. 4 is an oblique perspective drawing of the support equipment for measuring optical nerve-activity with a couple of the main bodies. The support equipment for measuring optical nerve-activity in this embodiment has the pair of main bodies 20a, 20b. Structures of the main bodies 20a, 20b are the same as that of the previously-described-embodiment except that the light emission region 23a for irradiating the light to one of the subject eyes is set at one of the main bodies 20a and the light emission region 23b for irradiating the light to the subject-other eye is set at the other main body 20b.

As shown in the FIG. 4, the main bodies 20a, 20b are layered keeping the light transmission directions same. In this case, they are layered with one of the main bodies 20a being shifted from the other 20b in the optical fiber 11-axis-direction. Thus, one ends of the layered main bodies 20a, 20b can be bundled only on one side of the subject-head region.

In the second embodiment of the present invention, the light leak parts 11c in one of the light emission regions may be positioned in every other optical fiber or every plural optical fibers alternately with those in the other light emission region as the previous embodiment. Moreover, in the second embodiment of the present invention, the right and the left-light emission regions 23a, 23b can employ the same light leak-part-positioning. For example, the plurality of light leak parts 11c may be positioned in a zigzag pattern as shown in the FIG. 5. For the sake of convenience also in the FIG. 5-preparation, the light leak parts 11c are positioned in a zigzag pattern in every other optical fiber in one of the light emission region 23a alternately with those in the other light emission region 23b. The light leak parts 11c may be positioned in a zigzag pattern in every plurality of the optical fibers, for example every 10˜20 optical fibers, in one of the light emission region 23a alternately with those in the other light emission region 23b.

### Another Embodiment

There is no specific drawing prepared for explaining another embodiment in the present invention. In another embodiment of the present invention, two or more of the main bodies 10 in the first embodiment or two or more of the main bodies 20a, 20b in the second embodiment are layered. Each of the light emission regions 13a, 13b, 23a, 23b is doubly or more layered in this embodiment of the present invention: therefore, the support equipment for measuring optical nerve-activity in the embodiment has an advantage over a situation such as using of one layer of the main body cannot provide enough lighting intensity. In this embodiment, as described in the second embodiment, the main bodies 10, or, 20a, 20b preferably are multiple-layered keeping the light transmission directions same so that one ends of the layered main bodies 10, or 20a, 20b can be bundled only on one side of the subject-head region.

The preferred embodiments of the present invention include the above described-preferred embodiments but not be limited to. For example, the above description that explained the main body is a cloth and the part of which is constituted by the optical fibers; however, the main body and the optical fibers are not necessarily combined as the one unit. If the main body is thin walled, lightweight, flexible, and has hard-to-rupture-solidity and it can couple the plural optical fibers, the optical fibers may be prepared as the one unit independent from the main body and the optical fibers may be fixed to the main body formed by resin film or paper (Japanese paper) and the like. In addition, the above explanation described the shape of the light emission regions 13a, 13b, 23a, 23b to be rectangular; however, their shapes may be made round or oval, which can provide the irradiation light at high lighting intensity-level efficiently preventing unnecessary light leak. Furthermore, the above explanation described that light is provided only from one ends of the optical fibers 11 by setting the connector 12a at one ends of the optical fibers 11; however, the light intensity level can be raised by setting the connectors 12a at both ends of the optical fibers 11 and providing the light from both ends of the optical fibers 11.

The support equipment for measuring optical nerve-activity in the present invention can be applied not only in neurosurgical operations under anesthesia but also any medical practices which require monitoring of optical nerve-activity by providing light stimulation for subject-eyes.

### BRIEF DESCRIPTIONS OF THE DRAWINGS

The FIG. 1 is an oblique perspective outline view for explaining whole structure of the support equipment for measuring optical nerve-activity in the first embodiment of the present invention.

The FIG. 2 is an enlarged cross section view of the main part of the optical fiber wherein the light leak parts are formed for showing ab example of the light leak parts.

The FIG. 3 is a plain view for showing an example of positioning of the light leak parts composing the light emission regions.

The FIG. 4 is an oblique perspective view of the main bodies of the support equipment for measuring optical nerve-activity in the second embodiment of the present invention.

The FIG. 5 is a plain view for showing an example of positioning of the light leak parts composing the light emission regions in the second embodiment of the present invention.

### EXPLANATION ON THE CHARACTERS

1: Support equipment for measuring optical nerve-activity
10: Main body
10a: Weft thread
11: Optical fiber (Warp thread)
12: Light emission device
13a, 13b: Light emission region
2: Support equipment for measuring optical nerve-activity
20a, 20b: Main body
23a, 23b: Light emission region

1. A support equipment for measuring optical nerve-activity used for measuring subject-optical nerve activity by providing light stimulation for the said subject eyes with a main body fixed to the said subject-head region, comprising:
the main body which is thin walled with a plurality of optical fibers joined in the width direction; and
light emission regions at least one of which is positioned in the said main body aligned to the said subject eye-position(s) and composed of a group of plural light leak parts formed in peripheral surfaces of the said optical fibers, and
a connector which is set in at least one ends of the said optical fibers for connecting with a light emission device providing light for each of the said plurality of optical fibers.

2. The support equipment for measuring optical nerve-activity as in claim 1 wherein the said optical fiber-diameter is 0.5 mm or less, the thickness of the said main body including the said optical fibers is 1 mm or less, and the said main body-weight is 50 g/m or less.

3. The support equipment for measuring optical nerve-activity as in claim 1 wherein warp threads composing a cloth in the longitudinal direction which forms the said main body are partially or all replaced with the said optical fibers.

4. The support equipment for measuring optical nerve-activity as in claim 1 wherein the said light leak parts are composed of acute angle-incisions formed in the said optical fiber-peripheral surfaces at various orientations and intervals.

5. The support equipment for measuring optical nerve-activity as in claim 1 wherein one ends of the said optical fibers are bundled, melted, and cut.

6. The support equipment for measuring optical nerve-activity as in claim 1 wherein the said light leak parts in a plurality of the optical fibers arranged in parallel in the said-single main body are formed in every other or every plurality of the optical fibers in one of the said light emission regions alternately with those in the other light emission region.

7. The support equipment for measuring optical nerve activity as in claim 1 wherein a plurality of the said main bodies are layered keeping light propagation directions in the said optical fibers same.
